(19) United States
(12) Patent Application Publication
(10) Pub. No.: US 2025/0329416 A1
(43) Pub. Date: Oct. 23, 2025

(54) Systems and methods for performing Axiomatic Ancestral Stratification by Kinship

(71) Applicant: Arun Christopher Konanur, London (CA)

(72) Inventor: Arun Christopher Konanur, London (CA)

(21) Appl. No.: 18/641,045

(22) Filed: Apr. 19, 2024

(51) Int. Cl. G16B 30/10; G16B 10/00; G16B 50/30

(52) U.S. Cl. CPC G16B 30/10; G16B 10/00; G16B 50/30

(57) ABSTRACT

A bioinformatic system that identifies the common ancestral origins of minimally correlated autosomal DNA (atDNA) matches is disclosed. The invention consists of three main components: The first is Axiomatic Ancestral Stratification by Kinship (AASK) a process of collating a collection of atDNA matches along ancestral family lines in order to establish a hierarchical sense of their common pedigree. The second is a set of automated scripts, formulae, and data structures to facilitate desktop correlation and tabulation utilizing AASK in conjunction with a desktop spreadsheet program such as Microsoft Excel. The third is a system of data tables and methods to facilitate AASK within a database management system (DBMS) at the enterprise level.

### FIELD OF THE INVENTION

The present invention relates to a system that performs Axiomatic Ancestral Stratification by Kinship (AASK), a method of organizing autosomal DNA matches, both on a personal (desktop spreadsheet tabulation) and on an enterprise (database management system) platform.

### BACKGROUND OF THE INVENTION

Direct-to-consumer autosomal DNA (atDNA) testing for the purpose of ancestry analysis was introduced in 2007, and since then millions of consumers have purchased test kits from one or more commercial entities which offer this service (23andMe, AncestryDNA, Family Tree DNA, MyHeritage, etc.). In each case, an individual's atDNA is sampled along roughly 700,000 single-nucleotide polymorphisms (SNPs), which are in turn compared against the test results of other customers of that same service (as many as 25 million other tests depending on the service), in order to generate a list of member matches—generally presented as a list of member names and/or test kit numbers. This list of member matches may consist of anywhere from several hundred names/subject identifiers to more than 100,000 such matches, depending on the results of the subject's DNA test, the prevalence of genetically related subjects already tested, and the degree of endogamy present in the subject's ancestral or ethnic subgroup.

Correlated Multiphasic Analysis (CMA) (U.S. application Ser. No. 17/470,321), a bioinformatic system that identifies the common ancestral origins of otherwise uncorrelated autosomal DNA (atDNA) matches, delivers powerful insights drawn from the totality of a subject's atDNA results. The end product of CMA is a collection of individuals/identifiers connected to a nexus individual through the pedigree and relations of a designated “Target Ancestor” of that nexus. CMA may yield a collection of anywhere from several hundred to several thousand elements—actionable intelligence, to be certain, culled from potentially millions of DNA matches—but a collection nevertheless too large and diffuse for directed investigation.

The purpose of AASK is to reveal the latent ancestral origins of genetic complexes defined by CMA, to partition these sets into collections of DNA matches that share a common ancestral line of descent, and to organize these lines into a hierarchical structure that reflects the degree to which each line of descent is more or less closely related to the Target Ancestor through which all such lines are connected. This hierarchical arrangement facilitates directed investigation through traditional genealogical methods and practice: building up family trees for individual subjects, discovering common surnames and ancestors, and connecting outliers to a common hierarchy by utilizing statistical methods based on the probabilities implicit in varying amounts of shared atDNA.

Traditional investigative methodologies are often hampered by non-existent or otherwise inaccurate pedigrees created by novice researchers who may have only recently begun to document their lineage. AASK avoids these pitfalls by employing an exclusively set-theoretic approach which does not require any degree of 3^{rd} party involvement or collaboration beyond providing access to the DNA matches themselves.

### SUMMARY OF THE INVENTION

This invention is directed to both refine and extend the usefulness of the CMA process by taking as its input a CMA-defined genetic complex, stratifying that complex into subsets consisting of DNA matches sharing a single ancestral line of descent, and then further organizing those subsets into an ancestral hierarchy based on the degree of set-theoretic inclusion exhibited by these subsets.

Unlike CMA, which presents the researcher with a wealth of analytic choices through which to organize and filter data, AASK is essentially a “black box” process: once its inputs have been loaded, AASK requires no user assistance or intervention to produce its hierarchical output. AASK employs several parameterized settings which may be adjusted to provide optimal results with larger or smaller datasets, or to allow for some degree of compatibility with endogamous populations and/or instances of pedigree collapse.

As with CMA, when deployed at the enterprise level, AASK leverages large sets of atDNA matches, and does not require associated family trees. AASK does not require additional processing of raw atDNA data, nor does AASK assume any advanced scientific knowledge on the part of the end user. In the course of its operation, AASK performs basic preprocessing of its data inputs in order to ensure the integrity of its operation and to minimize trivial findings.

Although AASK was initially developed to extend the utility of CMA, in practice CMA itself functions as something of a “pre-process” for AASK: filtering inputs and ensuring that AASK's findings are organized around a selected “Target Ancestor.” Given sufficient computing resources, however, AASK itself may be deployed to organize the entirety of an individual's autosomal DNA matches—especially useful in the context of adoptees and in cases where an individual might have no indication whatsoever as to the identity of a missing parent or grandparent.

### BRIEF DESCRIPTION OF THE DRAWINGS

In order to facilitate a fuller understanding of the present invention, reference is now made to the accompanying drawings. These drawings should not be construed as limiting the present disclosure, but are intended to be exemplary only.

FIG. 1 is a process flowchart illustrating Axiomatic Ancestral Stratification by Kinship (AASK) from the perspective of the end user. Sub-processes and connections to other figures have been numbered for reference; references are maintained throughout the detailed description of the invention.

FIG. 2 illustrates the data inputs required by AASK and their location within the desktop VBA implementation of AASK, the AASK Engine.

FIG. 3 presents an overview of the computational structure of AASK. An inset diagram reveals the hierarchical shorthand used to organize AASK's output.

FIG. 4 illustrates the preliminary preprocessing of data inputs performed by AASK, the parametrization of certain global settings, and the initialization of data tables.

FIG. 5 illustrates the method by which AASK gathers elements of its source dataset into a-classes: collections of individuals that share a common line of descent.

FIG. 6 illustrates the method by which AASK processes the elements of each a-class into β and γ collections.

FIG. 7 illustrates the method by which AASK creates an ordered intermediary meta-class, the delta-set, and assigns a hierarchical positioning vector (sn) to its y-classes, beginning with class 0 and the *-class.

FIG. 7a illustrates the method by which AASK assigns a hierarchical positioning vector (εₙ) to its y-classes, employing an iterative variant of the process of FIG. 7.

FIG. 8 illustrates the process by which AASK formats its output as an interactive Tree Report and printable Ancestral Stratification.

FIG. 9 presents the interactive Tree Report.

FIG. 10 presents the printable Ancestral Stratification.

FIG. 11 illustrates a sample CMA scenario, with five (5) descendants of a Target Ancestor.

FIG. 12 illustrates a CMA-derived genetic complex of around 800 individuals.

FIG. 13 illustrates the unknown pedigree of a Target Ancestor.

FIG. 14 illustrates the transmission of DNA inherited by descent with inheritance vectors.

FIG. 15 illustrates the inheritance vectors which connect elements of [symbol] with our Target Ancestor via Most Recent Common Ancestral Couples (MRCACs).

FIG. 16 illustrates the set-theoretic principle of inclusion and the manner in which AASK rescales the symmetric intersection of two collections to more accurately reflect their relationship.

FIG. 17 illustrates the MRCAC connecting individual [Mardell]₄ with the Target Ancestor.

FIG. 18 illustrates that the MRCAC's DNA is passed to descendants of the MRCAC.

FIG. 19 illustrates the pedigree of the MRCAC of individuals sharing [Mardell]₁'s line of descent, and the extent to which this pedigree includes the MRCACs of other ancestral lines.

FIG. 20 illustrates the symmetric nature of the first (intersection) table of the AASK Matrix.

FIG. 21 presents the table of FIG. 20, rescaled by |B|.

FIG. 22 contrasts the architecture of the Beta-build worksheet with that of the CMA Master Workbook.

FIG. 24 explores the mechanics of the process of FIG. 7a.

FIG. 25 presents the operational and computational tables employed by AASK's Hierarchy Matrix.

FIG. 26 presents an overview of the database tables required to implement AASK in a DBMS environment.

### DETAILED DESCRIPTION OF THE INVENTION

### I. The AASK Process

### Origins in CMA

Axiomatic Ancestral Stratification by Kinship (AASK) represents an outgrowth of the concepts and practices employed by CMA, and as such it may be helpful at the outset to review the CMA process.

In brief, CMA applies set-theoretic operations—primarily union (∪), intersection (∩), and complementation (˜)—to a core set of In Common With (ICW) atDNA matches to derive a genetic complex ( [symbol] ) genealogically related to our test subjects through the pedigree of a [symbol] selected “Target Ancestor.” CMA takes as its inputs the atDNA matches of a focal subject—designated as the nexus of the CMA process—and applies set-theoretic operations on this collection of DNA matches using the atDNA matches of established genealogical relations of the nexus.

By selecting appropriate test subjects culled from these genealogical relations, the end user may use CMA to derive a genetic complex ( [symbol] ) of DNA matches related to the nexus individual through the ancestors of a Target Ancestor whose pedigree is nonexistent or otherwise poorly documented.

The products of CMA which carry over into AASK are:
The genetic complex ( [symbol] ) of In Common With atDNA matches shared by the nexus and other direct descendants of a “Target Ancestor” which are subsequently refined by the CMA process. Within AASK, this set of DNA matches is known as complex-zero ( [symbol] ₀). The genetic complex derived by CMA may typically contain anywhere from a few hundred to a few thousand individuals.
The nexus and other individuals descended from the Target Ancestor.

These individuals are known as the generative elements (∈_{g}) of [symbol] ₀—even though, owing to the nature of CMA, these individuals are not themselves elements of [symbol] ₀. Since [symbol] ₀ is derived from the In Common With matches of the generative elements, we can write: [symbol] ₀⊆CW(∈_{g0}).

In short: CMA assembles a set of In Common With matches from a collection of generative elements, and then filters those ICW matches to arrive at a desired genetic complex. AASK, in turn, begins with that same genetic complex, and uses ICW matches derived from elements of the genetic complex in order to partition and hierarchically organize its genetic complex into collections of individuals sharing a common ancestral line of descent—the same sort of relationship shared by our original generative elements with the Target Ancestor.

The third input required by AASK are the autosomal DNA matches of each individual element of [symbol] ₀—which is to say that if [symbol] ₀ itself contains 200 individuals/elements (| [symbol] ₀|=200) then AASK requires 200 complete sets of atDNA matches in addition to [symbol] ₀ and the generative elements of [symbol] ₀ (∈_{g0}). The desktop VBA prototype of the AASK Engine can accommodate 3,000 distinct sets of DNA matches of up to 100,000 elements each, allowing the desktop prototype to analyze as many as 300 million points of data.

### AASK'S Use of Meta-Classes:

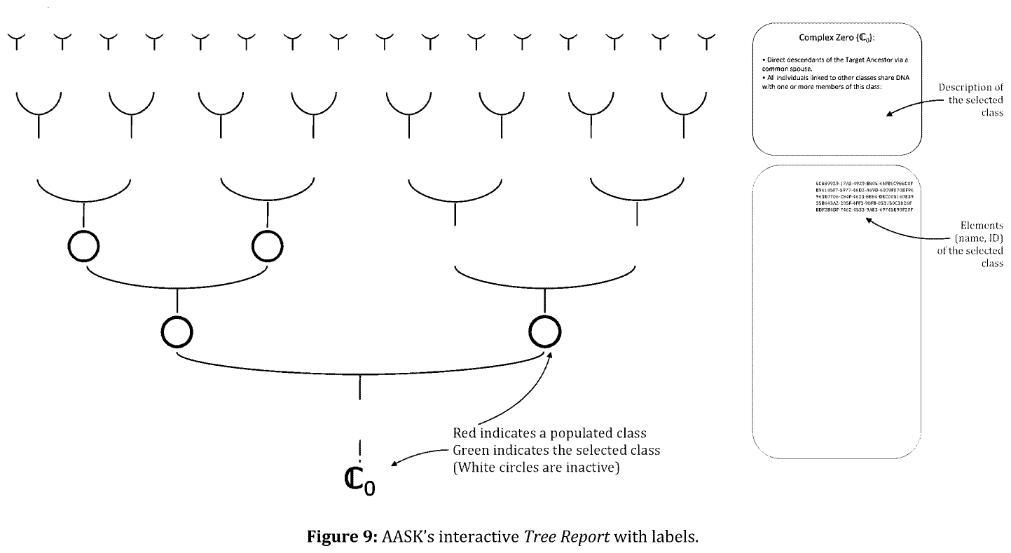

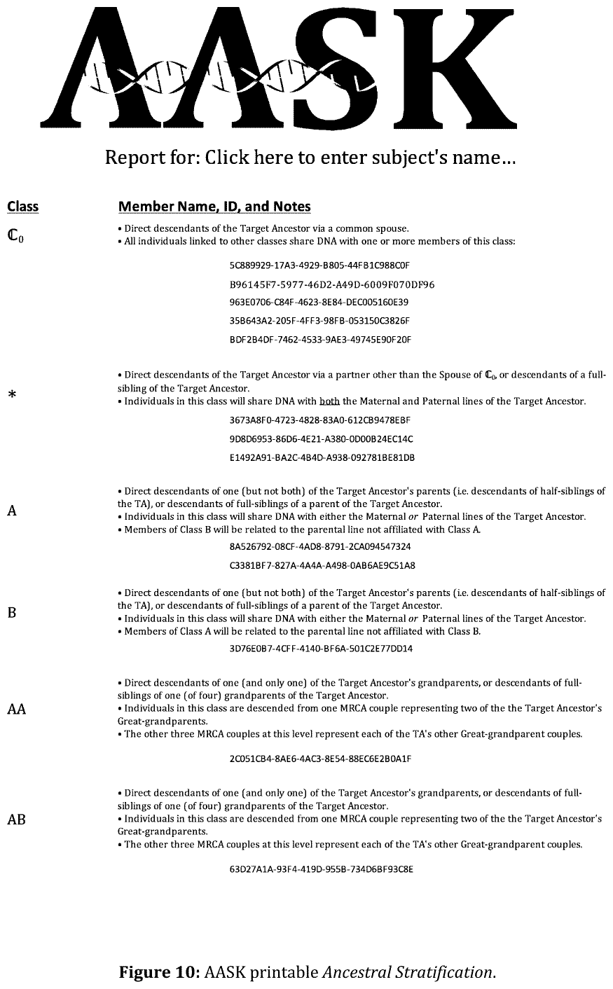

AASK organizes the constituent elements of [symbol] ₀ into meta-classes (or purpose-built subsets of data), which in turn are used to derive additional meta-classes in order to facilitate the partition and re-integration of [symbol] ₀ into a hierarchically organized whole:
To begin, AASK partitions [symbol] ₀ into alpha-classes (αₙ) which contain elements of [symbol] ₀ that share a common line of descent—which is to say that individuals with a given alpha-class are genealogically related to the generative elements of [symbol] ₀ through some common ancestor of the “Target Ancestor” which ∈_{g0} share. Since [symbol] ₀ is our source set, and because the generative elements of [symbol] ₀ already share a common line of descent (as these individuals are all descended from our Target Ancestor) we can designate the generative elements of [symbol] ₀ as our α₀.
The In Common With matches of each alpha-class (ICWαₙ) are tabulated to form a “beta-class” (βₙ). In the case of α₀, the generative elements of [symbol] ₀ form a genetic complex greater than that of [symbol] ₀ in isolation, so [symbol] ₀ is a subset of its beta-class ( [symbol] ₀ ⊆β₀).
AASK constructs a “gamma-class” (γₙ) from the intersection of each βₙ with [symbol] ₀. Since [symbol] ₀⊆β₀, γ₀=β₀ ∩ [symbol] ₀= [symbol] ₀. (one can appreciate how the ₀-instances of each class represent the identity or “universal-set” for our meta-classes).
“Delta-classes” (δₙ) are assembled by surveying the degree to which a given gamma-class includes the elements of each alpha-class that has yet to be assigned a hierarchical position within the Target Ancestor's pedigree. Unlike the preceding meta-classes, which are unordered collections, a delta-class is an ordered collection (an ordered binary, if you will) expressing in a Yes/No manner whether its associated gamma collection includes any of the generative elements (αₙ) of the unassigned gamma collections. Because the ICW matches of the generative elements of [symbol] ₀ populate each and every one of the gamma collections, the delta-class associated with γ₀ is δ₀={Y, Y, Y, Y, Y, . . . Y} where the number of elements in each delta-class is equal to n (the number of alpha-classes partitioned from the elements of [symbol] ₀) plus one, as the first term in the series represents whether γ₀ includes any elements of α₀. (And yes, every gamma-class includes its own generative elements).
The delta-classes are in turn evaluated in order to assign a “Hierarchical Positioning Vector” or epsilon-class (εₙ) to each α/β/γ/δₙ. The Tree Report (FIG. 9) diagrams this positioning vector, which is largely an ordered sequence of A and B (with notable exceptions to be outlined herein). As the Universal Set, δ₀ is assigned an ε₀ value of 0, representing the collection at the bottom of the hierarchy.

Prior to delving into the mechanics of the AASK Engine, it may be useful to clarify the mathematical underpinnings of AASK's meta-classes, as an understanding of these data types is foundational to an evaluation of the mechanics of AASK and the AASK Engine.

### i) The Alpha-Class—(αₙ)

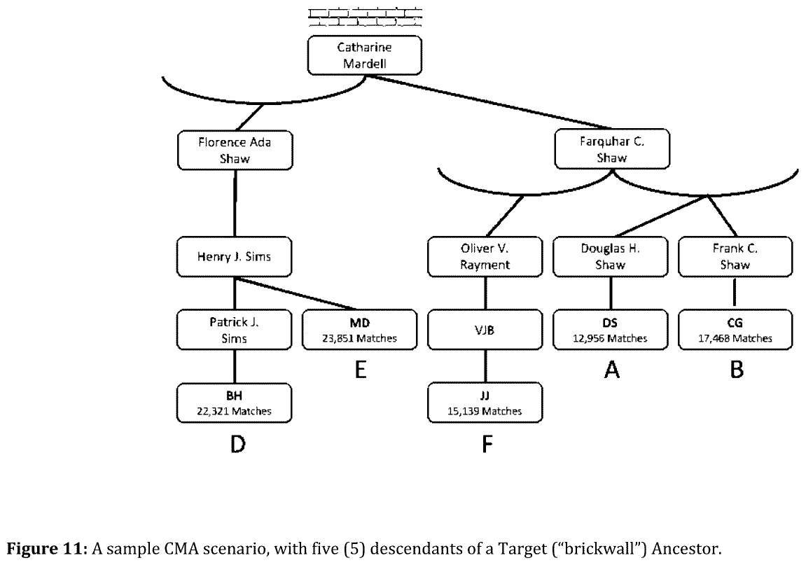

For reference: the CMA process filters the In Common With matches (i.e. the intersection sets) of the direct descendants of a “Target Ancestor.” FIG. 11 presents one such scenario, with five (5) descendants of a Target Ancestor (Catharine Mardell, b. 1839) whose uncertain pedigree is represented by a brick wall. Descendants A, B, and F share a common line of descent with regards to Catharine because their connection to Catharine's pedigree is through the same ancestor—Catharine's son Farquhar C. Shaw. Likewise, descendants D and E also share a common line of descent through Catharine's daughter Florence Ada Shaw.

From the perspective of Catharine's unknown pedigree, however, we may state that all five subjects (A, B, D, F, and F) all share a common line of descent, as their connection to Catharine's ancestry is through the same individual—namely Catharine herself.

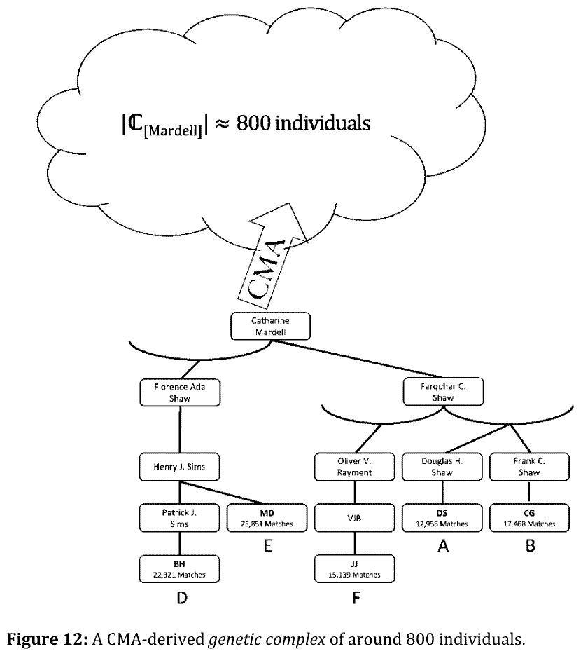

The set of DNA matches shared by two or more of our five subjects, filtered by CMA to remove connections to Catharine's husband's family lines, is an example of a genetic complex obtained through CMA—our [symbol] ₀—in which case our five subjects (A, B, D, E, and F) would be the generative elements (∈_{g0}) of our complex. Although [symbol] ₀ might contain any number of individuals, let us suppose that our CMA-derived genetic complex organized about Catharine ( [symbol] _{[Mardell]}) includes approximately 800 matches, as illustrated in FIG. 12.

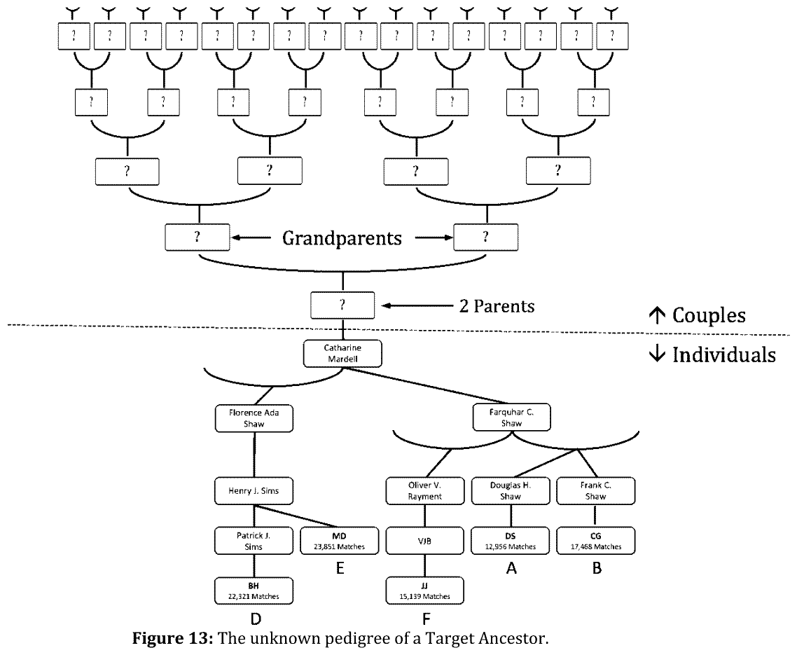

Although we have few (if any) specifics as to Catharine's pedigree, we can say with great certainty that she had 2 parents, 4 grandparents, 8 great-grandparents, 16 great-great-grandparents, 32 great-great-great-grandparents, and so on, through antiquity. FIG. 13 illustrates these unknown ancestral couples, each couple represented by a rectangle with a “?”

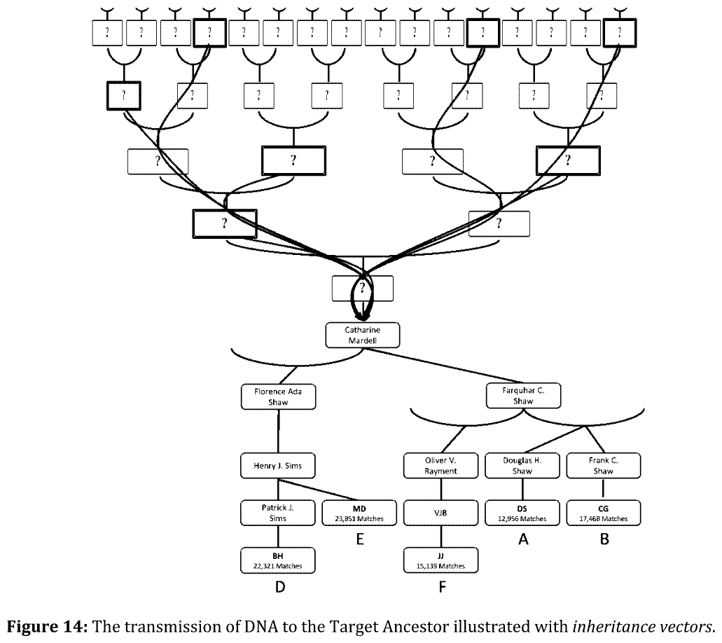

When we consider that the DNA Catharine has passed along to her descendants must originate from her own ancestors, we can conceptualize this genetic inheritance with vectored lines of descent originating from any given ancestor, passing through one or more generations of descendants, before arriving at Catharine, as illustrated by the inheritance vectors in FIG. 14.

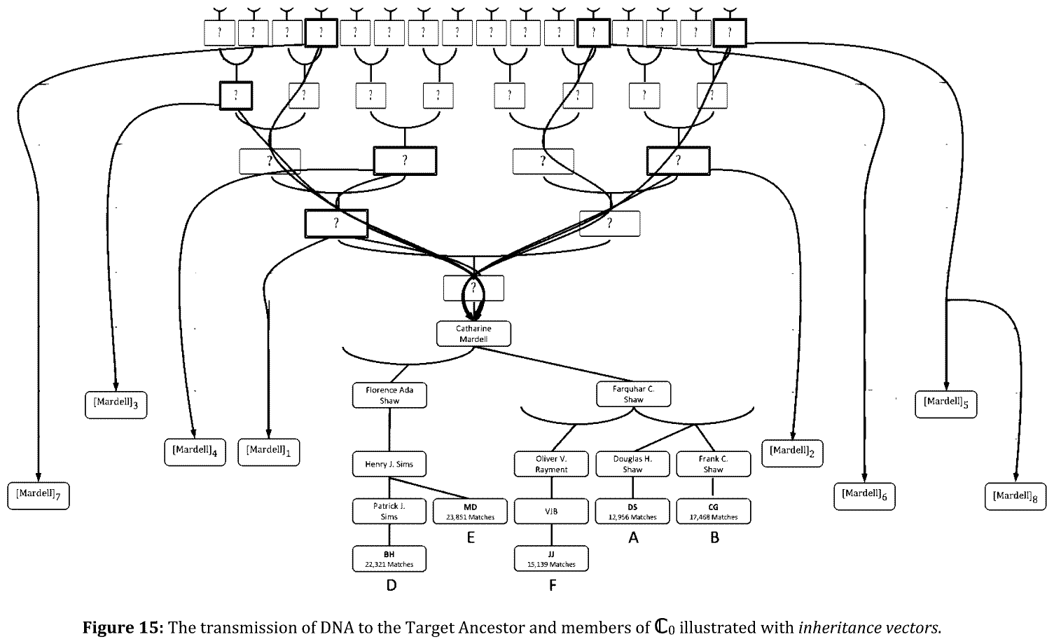

Further, if we acknowledge that each of the 800 DNA matches comprising [symbol] _{[Mardell]} share DNA with a subset of our generative elements—themselves descended from Catharine—then the elements of [symbol] _{[Mardell]} must also share one or more ancestral couples from Catharine's hypothetical pedigree. We can number the elements of [symbol] _{[Mardell]} as [Mardell]₁, [Mardell]₂, etc., and diagram possible inheritance vectors connecting these DNA matches to Catharine, as shown in FIG. 15.

FIG. 15 makes one thing abundantly clear: the limits of autosomal DNA testing necessitate that the 800 elements of [symbol] _{[Mardell]} cannot connect to Catharine through 800 distinct ancestors, and therefore must to some extent share ancestral lines of descent with one another with regards to Catharine's pedigree. In FIG. 14, Elements [Mardell]₅, and [Mardell]₈ share this type of relationship. Since a common line of descent defines the generative elements of our genetic complex [symbol] _{[Mardell]} and is a formative aspect of CMA, it follows that identifying similar collections within [symbol] _{[Mardell]} may hold the key to hierarchically organizing the 800 elements of [symbol] _{[Mardell]}.

Inasmuch as any individual element of [symbol] ₀ is unlikely to have inherited genetic connections to every relevant branch of the Target Ancestor's pedigree, the use of In Common With (ICW) matches from individuals sharing a common line of descent allows AASK to gather and assemble genetic information as comprehensively as possible.

Set Theory provides us with an effective indication as to which elements of [symbol] _{[Mardell]}share a given line of descent in the form of set-theoretic inclusion, which measures the degree to which distinct collections of elements mutually associate to form subsets. If we consider the DNA matches of each element of [symbol] _{[Mardell]} as separate collections of elements, we can assess the extent to which these collections include each other.

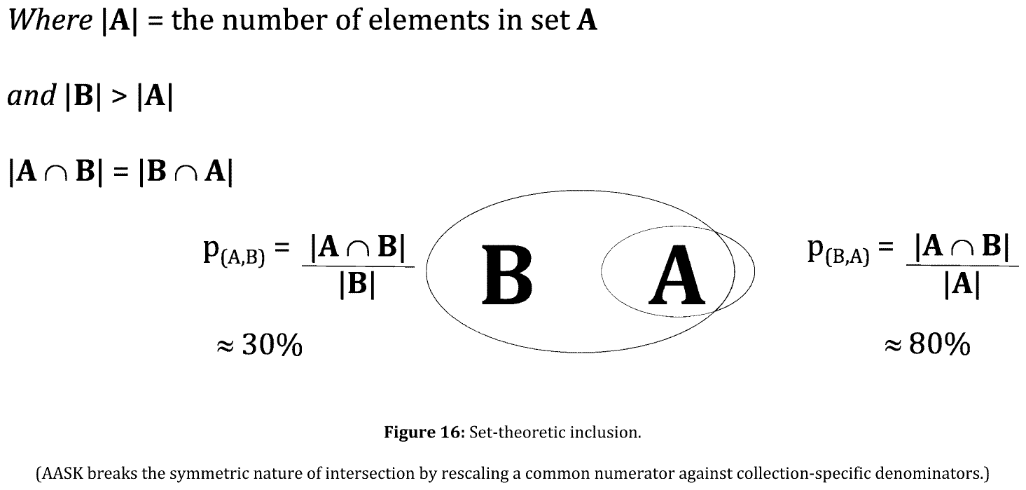

FIG. 16 presents two hypothetical sets A and B, where A is a proper subset of B. We can evaluate the |A∩B| to determine the number of elements shared by the two sets, which in the case of elements of [symbol] _{[Mardell]} would represent the number of DNA matches shared by two members of [symbol] _{[Mardell]}. However, because number of DNA matches for each individual can vary widely, and because |A∩B|=|B∩A| any meaningful measure of inclusion requires that we consider the number of shared matches in relation to an individual's total number of matches, and so we divide |A∩B| by the number of elements in the collection we wish to evaluate.

As such, FIG. 16 shows that collection A includes roughly 30% of collection B, whereas collection B includes roughly 80% of collection A. Therefore, where AASK is concerned, the measure of inclusion we must consider is one which rescales the magnitudes of its intersection sets to a percentage value of the collection as a whole.

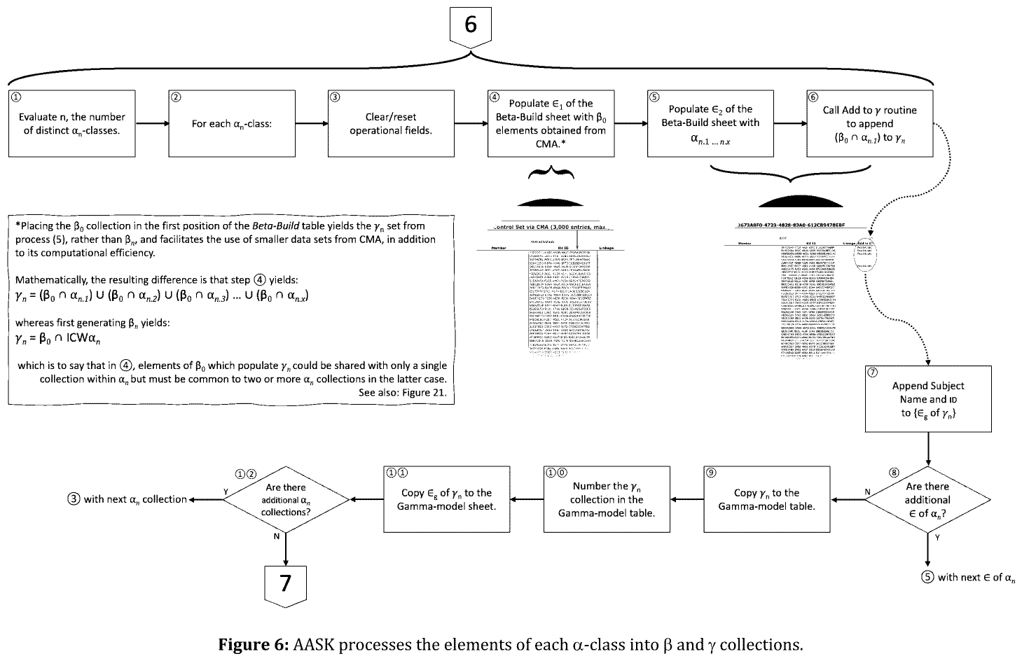

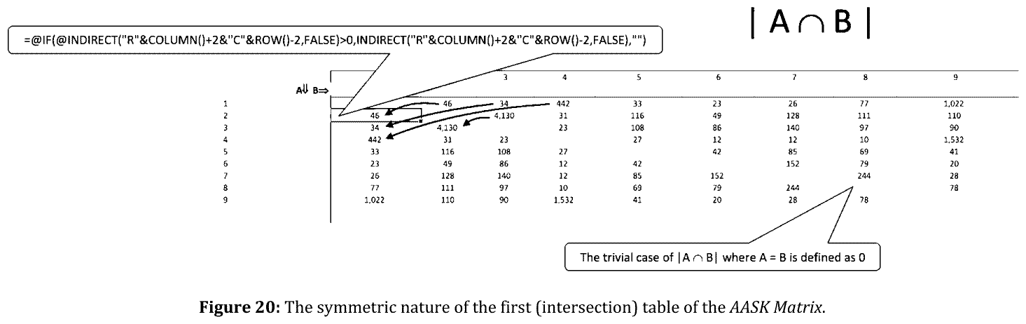

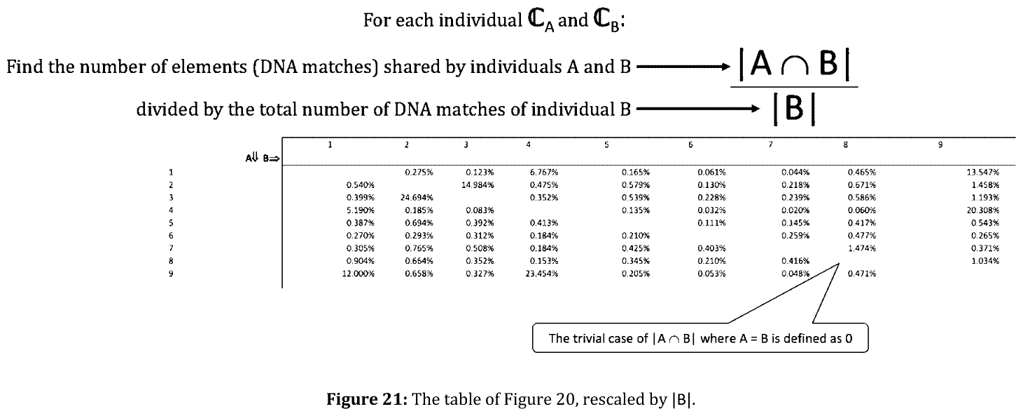

If we assemble a table of the degree to which pairs of elements of [symbol] _{[Mardell]} share their DNA matches (FIG. 20), and then rescale those values (FIG. 21) through the consistent application of the formulae of FIG. 16, we obtain a series similar to the 9-element sample which accompanies FIG. 5, process 2 (5.\textcircled{2}). AASK uses these values to identify which elements share the greatest percentage of their DNA matches with our test individual—and so it makes sense to sort these values from largest to smallest. (As the individual in question will always share 100% of its DNA matches with itself, the AASK Engine assigns this trivial relationship a value of zero in order to remove it from consideration.)

We can then evaluate the extent to which our percentage of shared matches declines from greatest to smallest; we do this by calculating the ratio between successive terms in our sorted series. As we are looking for the demarcation between two hypothetical collections—those elements of [symbol] _{[Mardell]} that share a common line of descent with our subject and those which do not—it follows that we should consider the significance of the largest ratio between successive terms, which would indicate that the terms preceding this large drop in shared matches have more ancestral lines in common with our subject and those which follow share less.

Of course, even if our subject is the only element of [symbol] _{[Mardell]} that shares its particular line of descent, there will still be a largest term in our series of ratios of sorted elements, and therefore it behooves our analysis to establish a floor below which a largest ratio value is no longer significant. This is the stratification ratio, whose value is set in 4.\textcircled{3}, and which may be parameterized to allow the AASK process to better adapt to analyze specific ancestral groups where endogamy may be prevalent.

The largest ratio among sorted elements, shown in 5.\textcircled{4}, is 18.2447405—which is indeed larger than the default stratification ratio of 5.0—so the elements which precede the ratio (in the example, [Mardell]₄, and [Mardell]₁) are grouped with [Mardell]₉ in a common instance of our lowest strata of meta-classes—the alpha-class—designated as an, where n is a non-zero whole number.

This process is repeated until all elements of our genetic complex have been assigned an alpha-class. (The AASK Engine is coded with provisions to append subsequent matches to an existing alpha-class should the need arise, but in principle this should be exceedingly rare).

### ii) The Beta-Class—(βₙ)

Following the form of CMA, which assembles a genetic complex organized about a common ancestral couple by tabulating the In Common With matches of a set of generative elements, AASK similarly regards the elements of each alpha-class (αₙ) as the generative elements of a line of descent and constructs a beta-class (βₙ) from the In Common With matches of its corresponding αₙ. This set of in Common With matches (ICWαₙ) is the set of all DNA matches shared by two or more elements of Un, and so necessarily includes ancestral lines not shared by our genetic complex. It is for this reason that βₙ represents an intermediary data structure, which is reconciled to our genetic complex with the next meta-class.

iii) The Gamma-Class—(γₙ)

CMA assembles a set of In Common With matches from its generative elements and subsequently uses set-theoretic operations to winnow that set down to the matches of our genetic complex [symbol] ₀. Similarly, AASK assembles an ICW set from the elements of each alpha-class and subsequently filters each beta-class by constructing γₙ from the intersection of βₙ with [symbol] ₀.

At this point it's worthwhile to consider just what each gamma-class represents in the “real world” and, by extension, what it does not represent. The gamma-class is a collection of DNA matches shared by the In Common With matches of a particular alpha-class and [symbol] ₀—but just what are these individual matches specifically?

Ordinarily, we might begin with the instance of γ₀, but since the generative elements of [symbol] ₀ are themselves not elements of [symbol] ₀, we must acknowledge that ∈_{g0}∉ [symbol] ₀. (This is because the generative elements of [symbol] ₀ are removed by CMA's set-theoretic winnowing of its genetic complex from a collection organized around an Ancestral Couple to a collection organized around a Target Ancestor). Paradoxically, since the generative elements of each gamma-class are themselves members of [symbol] ₀, [symbol] ₀ includes the generative elements of every γₙ except its own. For this reason, it's advisable to regard α₀ and instances of γₙ as a special case, as the elements of α₀ are to some extent represented in every γₙ.

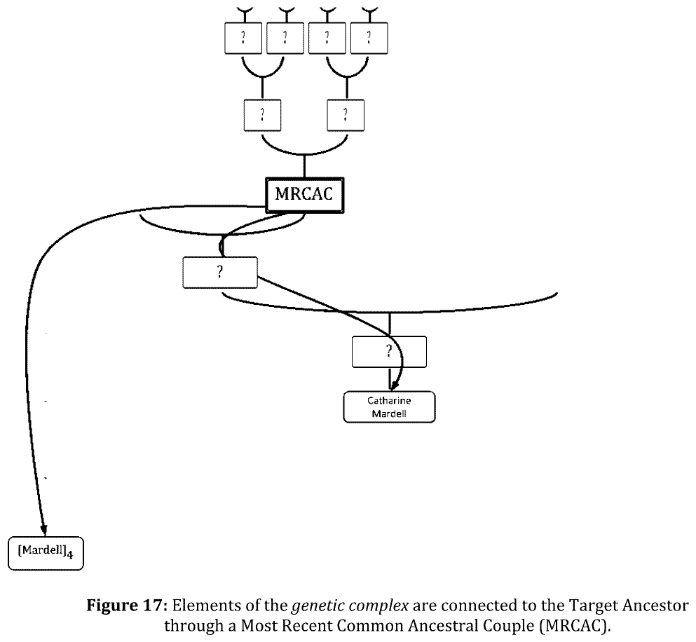

But what of the γₙ sets as a whole? Where do their matches originate, and how can we classify them? Let's consider the hypothetical instance of an element of [symbol] ₀ from FIG. 14, such as [Mardell]₄. FIG. 17 shows the Most Recent Common Ancestral Couple (MRCAC) connecting individual [Mardell]₄ and our Target Ancestor Catharine Mardell in isolation. The inheritance vectors illustrate how the MRCAC's DNA is passed along ancestral lines of descent to both Catharine and individual [Mardell]₄.

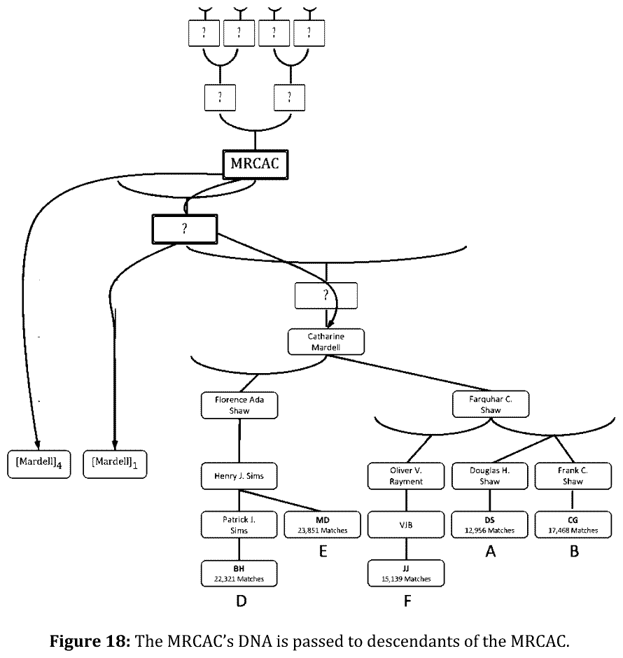

Our gamma-collection remains the set of DNA matches common to individuals sharing both [Mardell]₄'s line of descent and our genetic complex [symbol] ₀—so it's worthwhile to consider where else the DNA of our MRCAC goes. Obviously, the MRCAC's DNA is passed along to other descendants of that couple, such as [Mardell]₁, in addition to Catharine's descendants, as shown in FIG. 18. Although we would expect individuals descended from the unnamed ancestors of the MRCAC of FIG. 17 to match one or more individuals sharing [Mardell]₄'s line of descent, we would not expect any of the other [Mardell]ₙ individuals identified in FIG. 15, to match [Mardell]₄, with the exception of the line of [Mardell]₁ and Catharine's direct descendants (FIG. 18).

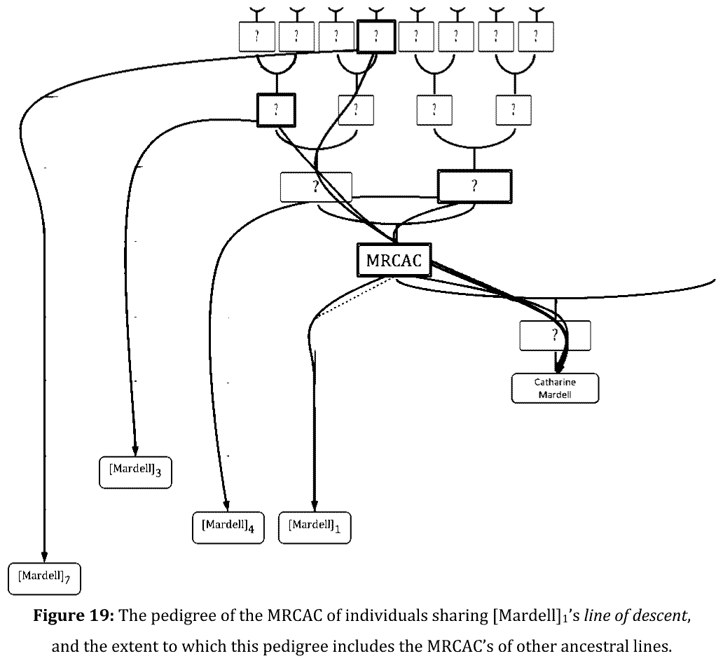

Likewise, if we were to consider the composition of the γₙ collection derived from [Mardell]₁'s line of descent, (FIG. 19) we would expect to find common DNA matches with the generative elements of the gamma collections of [Mardell]₄, [Mardell]₃, and [Mardell]₇—but not with the other ancestral lines of FIG. 15, except the generative elements of [symbol] ₀, which are common to all the gamma classes.

Since the gamma-classes of descendants of “downstream” MRCACs can include the generative elements of gamma-classes further “upstream”- and do not include matches with the generative elements of the MRCACs of other branches of our hierarchy—it follows that AASK should survey the extent to which the various gamma classes include the generative elements of the other gamma classes, working from the most inclusive collections (γ₀ includes the generative elements of all other classes) to the least inclusive. Further, since the absence of other lines of descent from our gamma-classes can be equally telling, the AASK process should also make note of the set-theoretic complements to a given gamma-class and the classes otherwise included within those complements, as this information also has a role to play assembling an ancestral hierarchy of gamma-classes.

### iv) The Delta-Class—(δₙ)

Each delta-class represents an aggregate snapshot of the degree to which its associated gamma-class includes, or does not include, the generative elements of other gamma-classes which have yet to be assigned a permanent positioning vector. For this reason, and in order to facilitate one-to-one comparisons with other delta-classes, the delta-class is an ordered set, where the number of elements in each ordered collection equals the total number of gamma-classes, preceded by an additional element that represents γ₀.

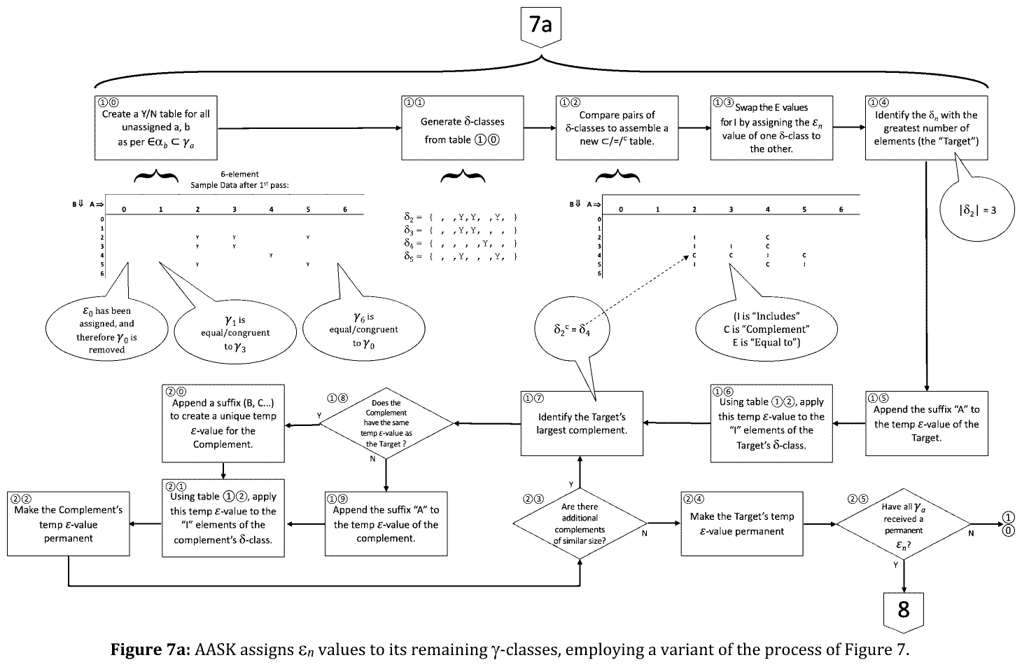

Because AASK employs a bottom-up methodology to assign each gamma-collection a position in the ancestral hierarchy, the set of delta-classes is re-evaluated after each generation of the ancestral hierarchy has been populated, in order to exclude already assigned gamma-classes from the next tabulation. The nature of this process will be made apparent in the course of examining the mechanics of the AASK Engine as presented in FIGS. 7 and 7a.

### v) The Epsilon-Class—(εₙ)

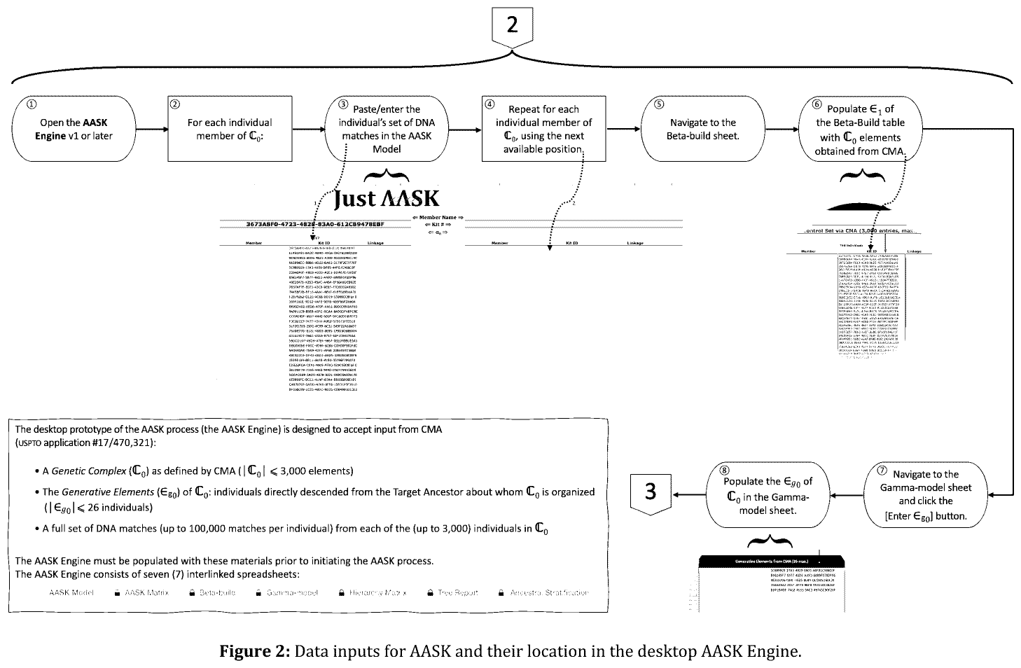

The AASK process reaches its apex with the epsilon-class, otherwise known as the hierarchical positioning vector. The value assigned to each εₙ has the effect of transforming a minimally correlated collection of individuals sharing a few common DNA matches into a hierarchically organized roster of subsets, each suited to further investigation through traditional genealogical investigative methods. AASK accomplishes this work without the benefit of preliminary investigative research, by utilizing the latent set-theoretic properties of the individual collections of DNA matches. AASK performs this work without user input, beyond supplying the raw materials enumerated in FIG. 2.

The Tree Report of FIG. 9 presents the range of values assigned to εₙ in an easily grasped hierarchy. As with the other meta-classes (where the generative elements of [symbol] ₀ give rise to meta-classes which also employ the 0 subscript) ε₀ is reserved for the δ₀ class. The various combinations of “A” and “B” which comprise the bulk of the εₙ values were selected because these two characters form what is essentially an “ordered binary”: an infinitely extensible system of either/or selections where the number of characters in an εₙ assignment indicates the number of generations the MRCAC of that class is removed from the parents of our Target Ancestor. The left-to-right ordering of the letters in a given εₙ assignment provides a navigable pathway through the hierarchy of generations, and the use of letters other than A and B provides AASK with methods for working with non-standard or incomplete hierarchies. (For example, if a genetic complex did not include any descendants of either set of the Target Ancestor's grandparents, then the εₙ values of “A” and “B” would remain unassigned. This would leave AASK with four (4) root-level classifications. However, because the classes “AA”, “AB”, “BA”, and “BB” imply a hierarchical grouping into (AA and AB) and (BA and BB) ancestral lines, AASK's strategy would be to label the lines as “A”, “B”, “C”, and “D” until further testing can augment and refine our genetic complex.

While the Tree Report resembles a traditional ancestral pedigree chart, important distinctions remain. For one, the chart does not identify the actual ancestors of our Target Ancestor in [symbol] ₀—the reason being that AASK's only data inputs are the names and identifiers of individuals who have taken a DNA test, and not the ancestors of these individuals. Second, each coded circle in FIG. 9 represents and ancestral couple rather than an individual.

Additionally, the A/B terminology of the Tree Report was selected precisely because these labels do not imply any type of gender bias or assignment: the “A” line of the chart may refer to the Target Ancestor's Maternal Grandparents or to the Target Ancestor's Paternal Grandparents. The only way to make such a determination is to take the individuals assigned to an “A” or “B” ε-class and “do genealogy”—building up the pedigrees of the individuals populating this class until we arrive at a common ancestral line that intersects with the times, places, and surnames of the Target Ancestor's pedigree.

In addition to the A/B positioning vectors, FIG. 9 presents two further classes at the bottom of the hierarchy: Class 0, assigned to the generative elements of [symbol] ₀, and the * class. The reason for these additional classes lies in the way CMA derives the genetic complex [symbol] ₀, and the dataset produced by that process. The generative elements of [symbol] ₀ produce a genetic complex organized around an Ancestral Couple: the Target Ancestor and whatever spouse is also common to our generative elements. CMA filters this collection so as to exclude DNA matches connected through the Target Ancestor's spouse, effectively creating a set of DNA matches connected to our generative elements through our Target Ancestor alone, which is to say a collection of DNA matches organized around a new Most Recent Common Ancestral Couple: the parents of our Target Ancestor, a collection which we refer to as [symbol] ₀. However, the direct descendants of our Target Ancestor are by no means the only individuals who might be related to our generative elements through the Target Ancestor's parents: children of the Target Ancestor with a spouse other than the one from which our generative elements are descended would qualify, as would the descendants of full-siblings of the Target Ancestor.

Unlike gamma-classes assigned to “A”, “B” or any combinations thereof, the In Common With matches of the generative elements assigned to the * class could potentially include the generative elements of all the other gamma-classes—much as γ₀ does. It is for this reason—the existence of a class that behaves like α₀ but isn't α₀—that the * class exists in the hierarchy. Of course, just because this positioning vector exists in theory doesn't mean that every [symbol] ₀ includes matches which satisfy the properties of the * vector: for instance, our Target Ancestor might have had offspring with only one partner and/or might have been an only child—or perhaps the descendants of the Target Ancestor's siblings have not yet tested—which is why the circle labelled with an asterisk (*) in the sample set of FIG. 9 is white (inactive).

The specifics of how En values are assigned is illustrated in FIGS. 7 and 7a and will be examined in detail in Section II, which explores the mechanics of the AASK Engine.

The following table summarizes AASK's meta-classes of data:

| | | | | 1:1 (com- |
|---|---|---|---|---|
| | | | Unordered | putation) or |
| | | | Collection or | Many to One |
| Name | Symbol | Definition | Ordered Set | (assigned) |
| Genetic | [symbol] 0 | Individuals selected | Unordered | Many:1 |
| Complex | | by CMA process | | |
| Alpha-class | αₙ | ∈ [symbol] ₀ sharing a | Unordered | Many:1 |
| | | line of descent | | |
| Beta-class | βₙ | ICW(αₙ) | Unordered | 1:1 |
| Gamma-class | γₙ | βₙ ∩ [symbol] ₀ | Unordered | 1:1 |
| Delta-class | δₙ | {∈ α_{b} ⊂ V γₙ} _{b0→n} | Ordered | 1:1 |
| | | (where ε_{b}, εₙ are Ø) | | |
| Epsilon-class | εₙ | Hierarchical | Unordered | Many:1 |
| | | positioning vector | | (potentially) |

### II. AASK on the Desktop Computing Platform Via the AASK Engine

The AASK process, as outlined in Section I, forms the theoretical basis for practical implementations of AASK on the desktop and enterprise platforms. A fuller understanding of the mechanics and particulars of AASK may be gleaned from an analysis of the AASK Engine, a desktop implementation of AASK in Microsoft Excel, scripted in Visual Basic for Applications (VBA). Version 1 of the AASK Engine is structured to organize up to 3,000 individual sets of DNA matches into as many as 255 instances of each of AASK's meta-classes of data. The AASK Engine's reports have been formatted to display 63 distinct meta-classes across 6 generations of the Target Ancestor's pedigree.

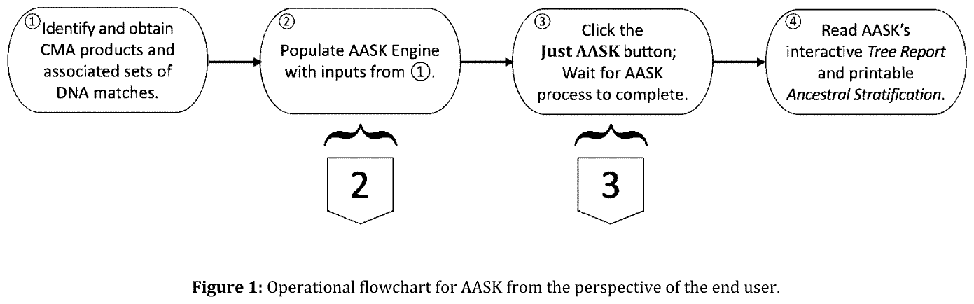

FIG. 1 is a process flowchart illustrating the end user's experience of the AASK Engine—the desktop prototype of AASK—which consists of a VBA scripted workbook assembled in Microsoft Excel. Each figure's sub-processes (1.\textcircled{1}, 1.\textcircled{2}, etc.) have been numbered for reference, and off-page connectors are numbered according to the figures to which they connect (2, 3, etc.).

FIG. 2 illustrates where AASK's data inputs are situated within the AASK Engine.

2.\textcircled{1}: The AASK Engine is a Microsoft Excel workbook consisting of seven (7) interlinked worksheets:
AASK Model
AASK Matrix
Beta-build
Gamma-model
Hierarchy Matrix
Tree Report
Ancestral Stratification

2.\textcircled{2}-\textcircled{4}: Sets of autosomal DMA matches for up to 3,000 test subjects (elements of [symbol] ₀) may be stored in the AASK Model. In addition to the Test Kit ID # of each match, the AASK Model also supports entry of each match's proper name and the linkage shared between the test subject (an element of [symbol] ₀) and its constituent matches. However, only the Test Kit ID # is required for AASK processing.

2.\textcircled{5}-\textcircled{6}: Position one (1) of the Beta-build worksheet accepts the Test Kit ID #s of the individuals which comprise the genetic complex [symbol] ₀, obtained via the CMA process. Although only the Test Kit ID #s are required, providing a proper name for each element of [symbol] ₀ at this location will carry the names of these individuals through to AASK's reporting modules, making the end user's reports that much easier to read and interpret.

2.\textcircled{7}-\textcircled{8}: The upper left quadrant of the Gamma-model worksheet has an [Enter ∈_{g0}] button, which brings the user to the area of the Gamma-model where the generative elements of [symbol] ₀ are entered. As with the elements of [symbol] ₀ itself, only the Test Kit ID #s are required, but providing proper names for each generative element will make AASK's final report that much more user-friendly.

3.\textcircled{1}: The [Just AASK] button initiates the AASK process and is located on the Gamma-model worksheet to the right of where the generative elements of [symbol] ₀ are entered and also in the upper left quadrant of the AASK Model worksheet. Either button may be used to initiate the AASK process.

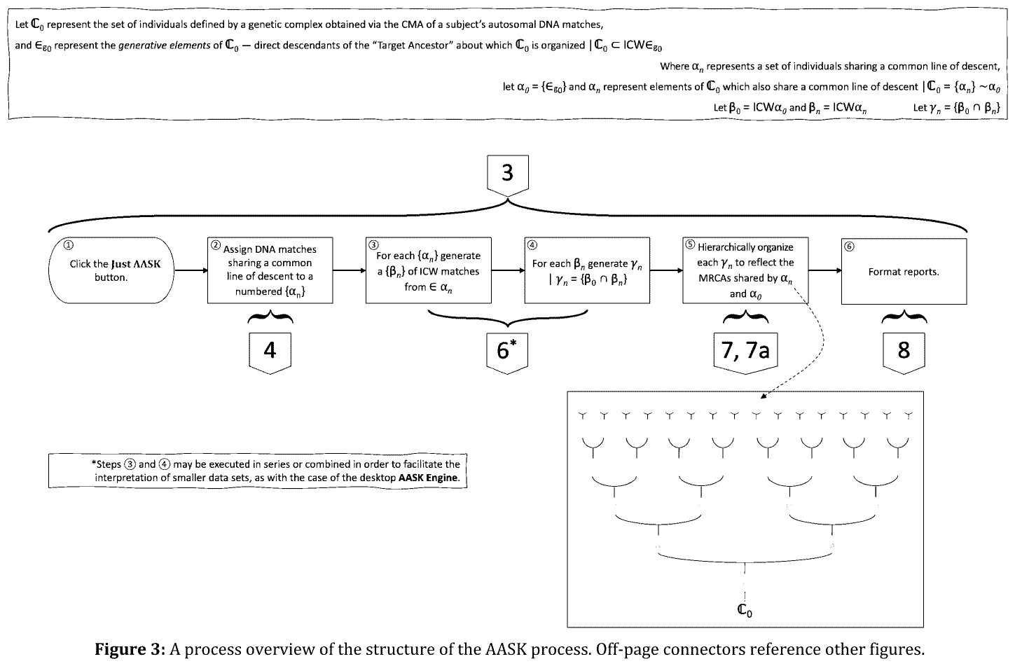

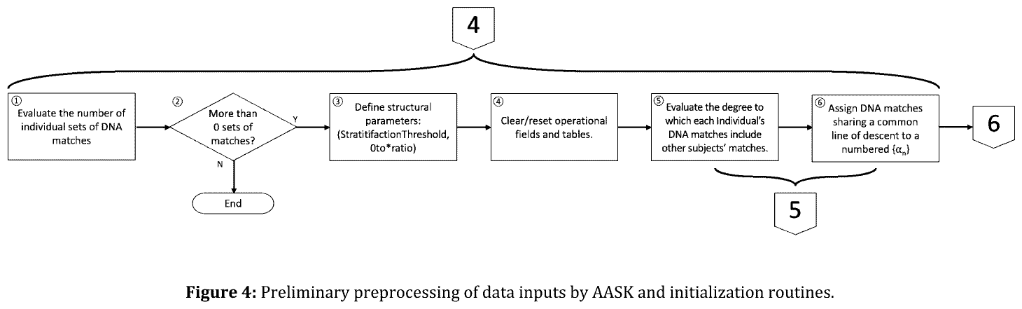

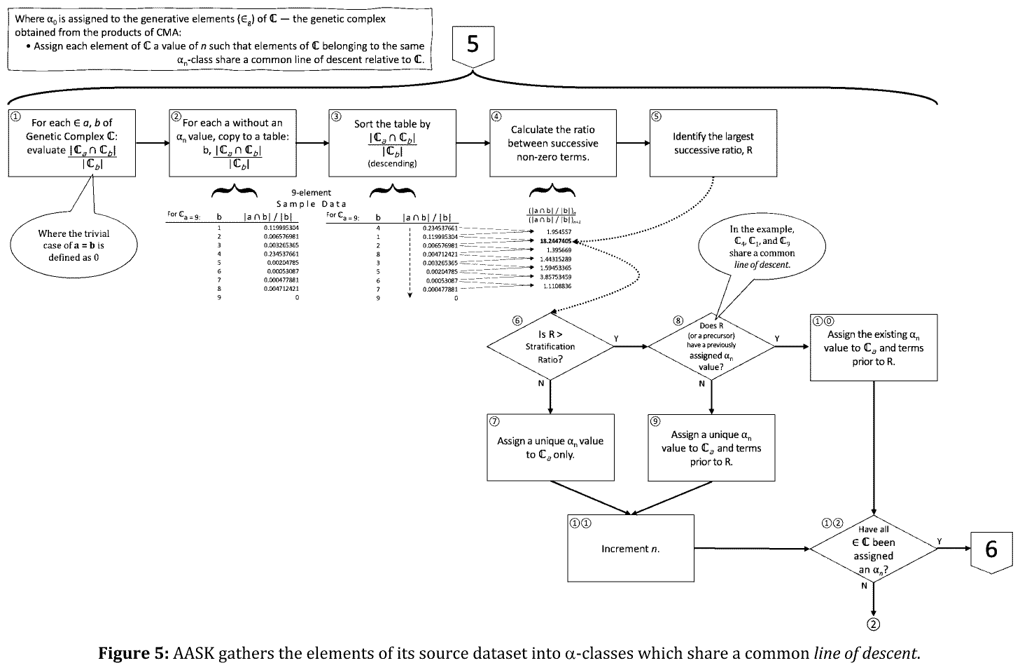

As the remaining processes of FIG. 3 reference other figures, they will be dealt with in detail as FIGS. 4 through 8 are discussed.

4.\textcircled{1}-\textcircled{2}: AASK begins by validating its inputs: verifying that the number of individual sets of DNA matches equals the number of elements of [symbol] ₀. The use of CMA to define [symbol] ₀ and its constituent elements has the effect of “pre-qualifying” AASK's inputs, but absent any disqualifying conditions (i.e. | [symbol] ₀|=0) AASK will proceed onwards. Other implementations of AASK may necessitate their own pre-qualification conditions, and those procedures would be coded here.

4.\textcircled{3}: AASK defines certain structural parameters as global variables, for example:
The Stratification Ratio (SR) is used to determine how elements of [symbol] ₀ are grouped into common lines of descent. This ratio is nominally set to equal 5. Higher values will yield more closely focused ancestral lines, useful when [symbol] ₀ is derived from an endogamous population. Conversely, lowering this value may be useful in situations where the Target Ancestor is many generations removed from the generative elements of [symbol] ₀, or possibly when many generations separate the generative elements themselves.
The 0to*ratio (Zero*) is used to determine the presence of the (*)-class when assigning Hierarchical Positioning Vectors to the εₙ-classes after the ε₀ class has been assigned. This value is nominally set to 0.75, but may be increased in cases where one (maternal/paternal) branch of the Target Ancestor's ancestral lines is grossly overrepresented.

4.\textcircled{4}: AASK employs interlinked data tables of two varieties: operational tables contain data populated by the AASK process—typically through iterative or looped processes. Computational tables are pre-populated with formulae which dynamically recalculate their values based on the references to other computational or operational tables. An example of each type of table is found in process 5.\textcircled{1}, but suffice to say that the AASK Engine only needs to clear out the remnants of data populated by prior executions of the process in order to begin with a “clean slate.”

The remaining processes of FIG. 4 are discussed in detail in FIG. 5.

5.\textcircled{1}-\textcircled{2}: AASK evaluates the degree to which elements of [symbol] ₀ include each other in the most computationally efficient manner possible. FIG. 20 illustrates the first table of the AASK Matrix worksheet. Since |A∩B|=|B∩A| the table of FIG. 20 is a hybrid construction of operational fields where B>A, mirrored with computational formulae where A>B. The trivial case of |A∩B| where A=B is defined as zero. This table extends for 3,000 elements on both axes.

The columns of FIG. 20 are rescaled by the number of DNA matches of individual B to produce the table of FIG. 21—the 2^{nd} table of the AASK Matrix. The values in the columns of this table provide a uniform basis from which to evaluate the degree to which the DNA matches of a given individual include the matches of the other elements of [symbol] ₀. Rescaling the columns of FIG. 20 allows for comparisons among meta-classes beyond those specifically required by the AASK process itself.

5.\textcircled{3}: Because individuals sharing a common line of descent will share numerous ancestral lines in addition to the line connecting with [symbol] ₀, we can sort these rescaled values from those with the greatest degree of inclusion to least.

5.\textcircled{4}-\textcircled{1}\textcircled{2}: The rescaled and sorted measurements of inclusion fall into two groups: DNA matches that share a line of descent with individual B and those matches which do not share common ancestral lines with B outside of [symbol] ₀. We could graph these values to visually determine where this separation occurs, but since AASK is a computational process, the simplest way to assess this division is to evaluate the ratios between successive terms in our sorted series. The largest ratio in our series (R) will represent the “dividing line” between DNA matches which share our subject B's ancestral lines beyond [symbol] ₀ and those which do not.

Recognizing that any and all series of sorted DNA matches will necessarily have a largest ratio among successive terms, AASK employs a Stratification Ratio (SR) as a method of filtering out values which might otherwise be erroneously included among a subject's line of descent. If the largest ratio among successive terms does not exceed the SR, then the subject B is assigned to its own exclusive ancestral line—the alternative (trivial/null) hypothesis being that all elements of [symbol] ₀ belong to the same line of descent. Otherwise, assuming R exceeds the SR, the terms which precede the largest ratio are assigned to the same line of descent as B. Each shared line of descent is represented by a common at, value assigned to the elements of [symbol] ₀ assigned to that class.

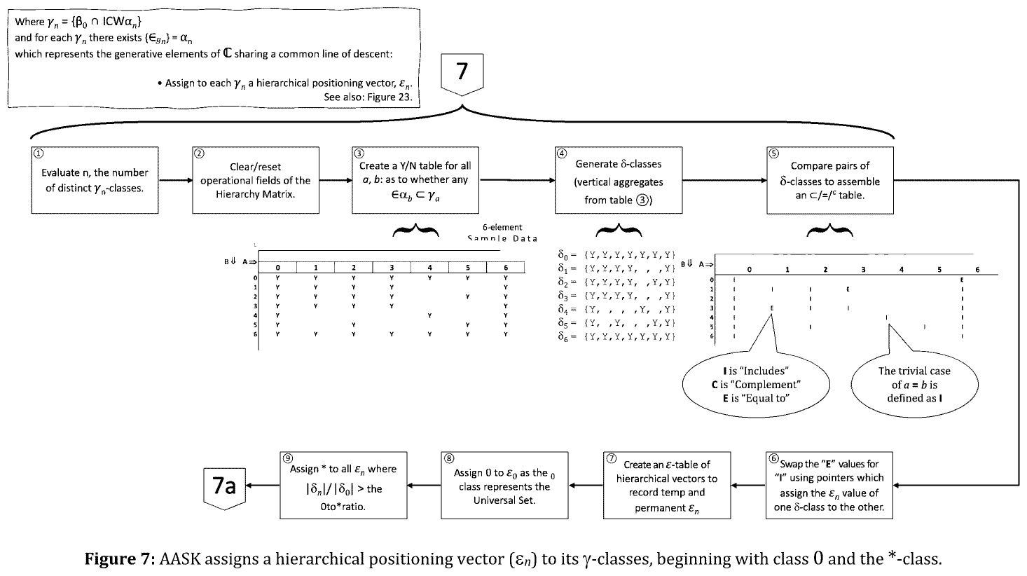

The process from 5.\textcircled{2} onwards is repeated until all elements of [symbol] ₀ have been assigned an αₙ designation, at which point the AASK Engine proceeds onwards to the operations of FIG. 6.

6.\textcircled{1}-\textcircled{2}: FIG. 6 uses the Beta-build worksheet to processes the elements of each αₙ-class into beta (βₙ) and gamma (γₙ)-class collections. The AASK process defines the beta-class (βₙ) as the In Common With (ICW) matches of the elements of its corresponding αₙ collection, and the gamma-class (γₙ) as the intersection of the βₙ collection with the source genetic complex [symbol] ₀.

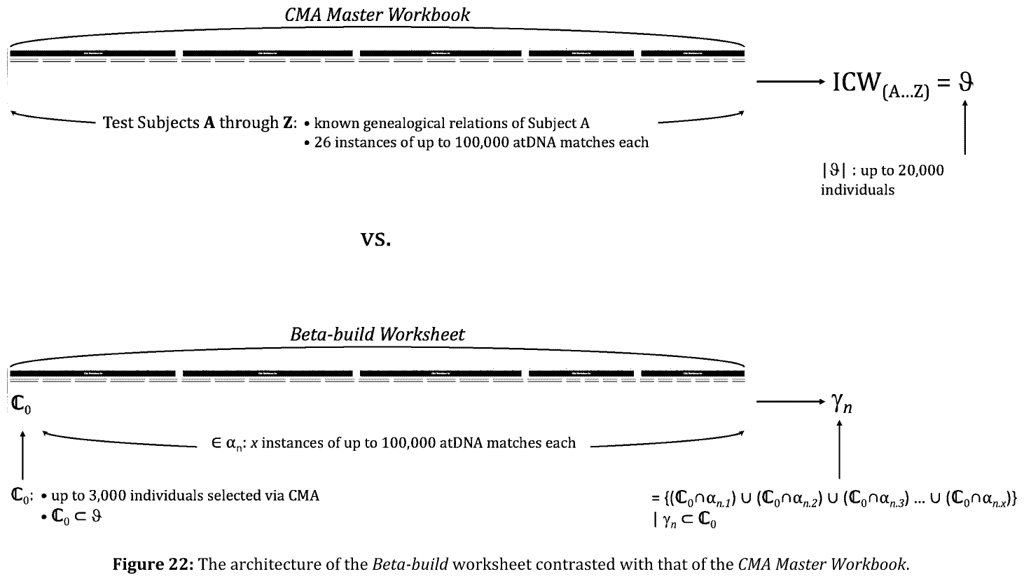

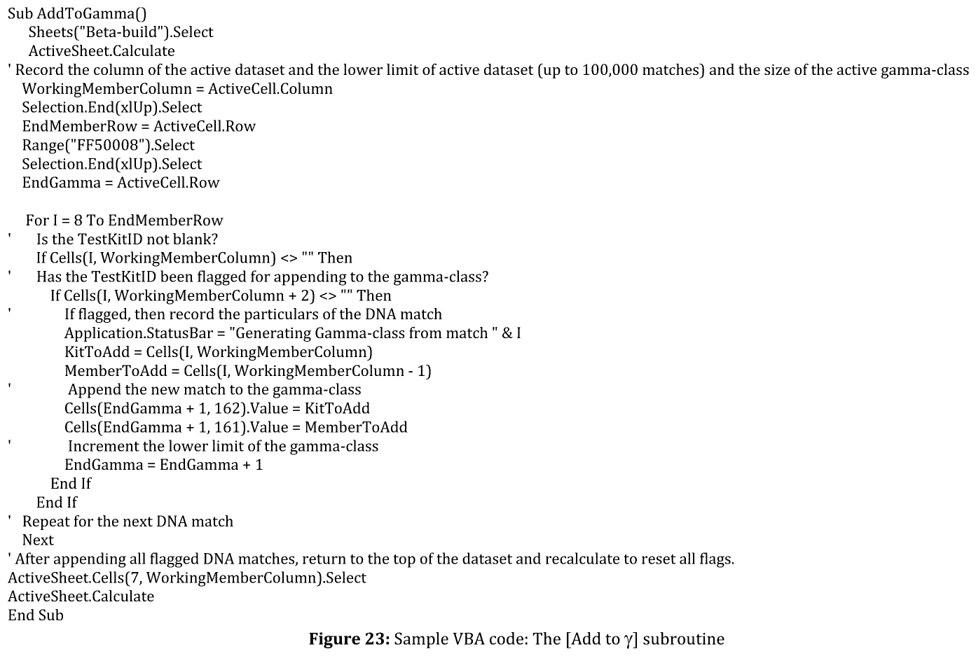

The Beta-build worksheet is modelled on the CMA Master Workbook in that the worksheet compares several secondary sets of DNA matches (the elements of a given αₙ class) against a reference set of individuals ( [symbol] ₀). However, whereas the CMA Master Workbook creates its Control Set by evaluating the In Common With matches of up to 26 individual sets of DNA matches, the Beta-build worksheet assembles its γₙ-class from the In Common With matches of a potentially unlimited series of elements of αₙ (up to 3,000 in the AASK Engine) compared against our reference genetic complex [symbol] ₀. FIG. 22 contrasts the construction of the two sheets.

6.\textcircled{3}-\textcircled{4}: Per FIG. 22, the [symbol] ₀/β₀ collection of individuals populates the leftmost dataset position of the Beta-build sheet (as per the data entry protocols of 2.\textcircled{6}). AASK clears the 2^{nd} dataset position.

6.\textcircled{5}: The 2^{nd} dataset position of the Beta-build worksheet successively accommodates the DNA matches of each element of the αₙ-class being processed by AASK. The formulae adjacent to each DNA match within the added dataset checks to see if that element is also found in [symbol] ₀ but not yet an element of γₙ. If both of these conditions are met, the element of the dataset is flagged for addition to γₙ.

6.\textcircled{6}: The [Add to γ] subroutine (FIG. 23, a variant of which was originally developed for the CMA Master Workbook) appends the DNA matches flagged in 6.\textcircled{5} for addition to γₙ.

6.\textcircled{7}: The Name and Subject ID of each element of αₙ processed by AASK is appended to a list of generative elements (∈_{g}) of that γₙ—irrespective of whether or not that individual directly contributed DNA matches to γₙ.

6.\textcircled{8}-\textcircled{1}\textcircled{1}: The process repeats from 6.\textcircled{5} onwards until the intersection of all αₙ with [symbol] ₀ have been evaluated, after which the assembled γₙ and its generative elements are copied to the Gamma-model worksheet.

6.\textcircled{1}\textcircled{2}: The process then repeats from 6.\textcircled{3} onwards, where the γₙ, ∈_{g}, and 2^{nd} dataset positions are all reset, n is incremented, and a new γₙ is enumerated.

The set of gamma-classes (γₙ) and their generative elements represent the raw materials from which AASK hierarchically organizes each gamma-class according to the scheme of FIG. 9. AASK does this by assigning every γₙ a hierarchical positioning ε-vector—typically consisting of combinations of the letters A and B. FIG. 7 illustrates the “first pass” of this process, which assigns a positioning vector of 0 to ε₀, the class associated with γ₀, and determines whether any ε-class is assigned the * value.

7.\textcircled{1}-\textcircled{2}: The number of gamma-classes (n) should be consistent with the number of instances of AASK's beta-classes; however, the AASK process verifies that this is the case. AASK clears the operational fields of the Hierarchy Matrix—namely the tables of 7.\textcircled{3} and 7.\textcircled{5} and the table of provisional and permanent ε-values.

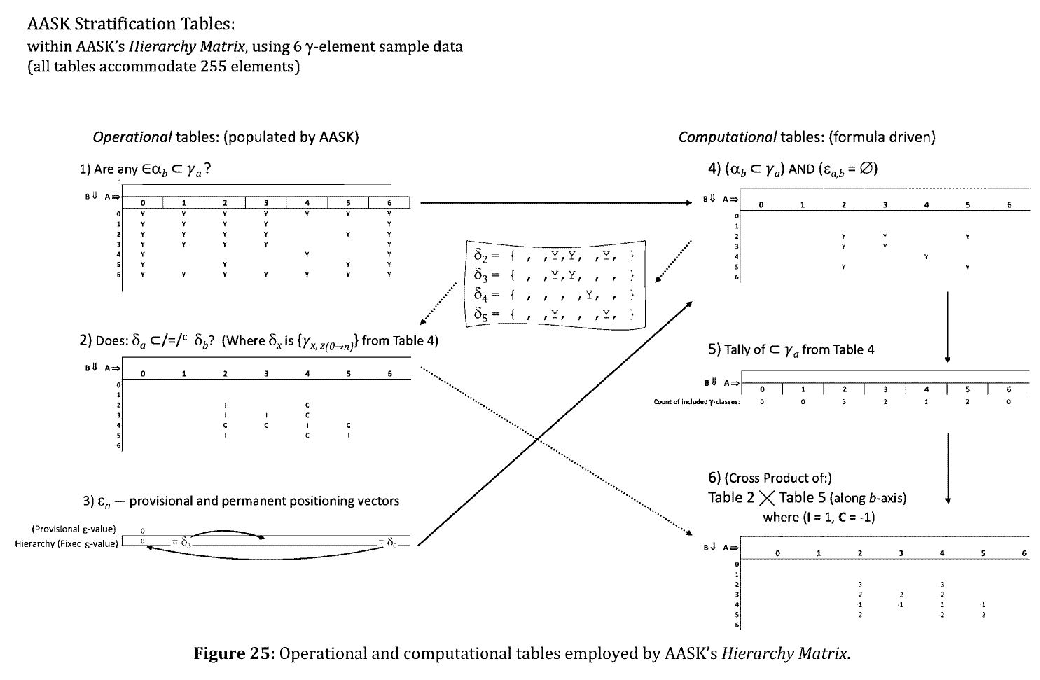

7.\textcircled{3}: An (n+1)-by-(n+1) truth table (FIG. 25, Hierarchy Matrix Table 1) is populated with the outcomes of: “does γₐ include any of the generative elements of γ_{b}?” (α_{b} ⊂γₐ). The trivial case of a=b is defined as Yes/True.

7.\textcircled{4}: Vertical aggregates (columns) from this table are taken to form delta-sets {δₙ}: ordered sets which tabulate whether any generative elements of the respective γₙ's are found within the elements of a given gamma-class.

As the first ordered element of these sets indicates the presence of the generative elements of γ₀—and since two or more of these elements are found in every γₙ—the first element of each of delta-set at this point in the AASK process is “Y”.

7.\textcircled{5}: Pairs of delta-sets (FIG. 25, Hierarchy Matrix Table 2) are evaluated so as to determine whether: δₐ includes δ_{b} (represented on the table by an I); is equivalent/congruent to δ_{b} (represented by an E); or whether δₐ and δ_{b} are complements, with no ordered elements in common (represented by a C). While complementation and congruence are symmetric functions, inclusion is not, so every pairing of a and b must be evaluated. The trivial equivalence of (a=b) is represented in this table by an I.

The presence of the γ₀ collection in the table at this stage of the AASK process ensures that no complementary sets will be tabulated here, as the first element of every set is “Y”.

7.\textcircled{6}: However, it is entirely possible that some pairs of delta-sets may be found to correspond precisely, and are labelled in the table with an E. These “congruent pairs” of delta-sets are by no means composed of identical collections of individuals, nor do they in actuality share identical lines of descent. Rather, it is the case that given the present composition of our CMA-derived genetic complex ( [symbol] ₀), AASK is unable to fully stratify/disentangle these equivalent collections. It is probable that with the genetic testing of additional individuals, [symbol] ₀ will be augmented with the addition of subjects whose lines of descent (and their respective generative elements) will differentiate these delta-sets. In reality, the MRCACs of this congruent pair may represent the maternal and paternal lines of an MRCAC otherwise unrepresented among the alpha-classes of [symbol] ₀.

AASK deals with this phenomenon by assigning (or “pointing”) the ε-value of one such delta-class to equal the value eventually assigned to the other class. As the delta-sets of these collections are identical, it does not matter which collection of the pair is referenced and which remains open for further evaluation. The principal effect of making this assignment is that it removes one congruent collection from evaluation during subsequent passes of the positioning vector assignment process. This reason, above all others, is why the trivial (a=b) congruence at this level is indicated by an I rather than an E.

The sample data presented in FIG. 7.\textcircled{5} has two such examples of congruence: δ₆ is congruent to δ₀, and δ₃ and δ₁ are congruent. This is to be expected, as six points of data are in no way sufficient to fully differentiate the elements of a genetic complex. AASK replaces the E at the top of column a=6 with an I, and enters a formula in the corresponding column of the ε-value table (see FIG. 25, Table 3) for δ₆, referencing whatever εₙ-class is eventually entered for δ₀ (δ₀ is always assigned ε₀). Similarly, AASK replaces the E within column a=1 with an I, and enters a formula in the ε-value table for δ₁ referencing whatever εₙ-class is eventually entered for δ₃. As such, the next iteration of this table (in FIG. 7.\textcircled{1}\textcircled{0}) does not tabulate data for values of a, b where a or b equal 1 or 6. Position 0 is likewise excluded, as the process of FIG. 7 assigns the value of zero to δ₀.

7.\textcircled{7}: AASK maintains an (n+1)-by-2 table of ε-values (FIG. 25, Hierarchy Matrix Table 3) which records a provisional and permanent ε-values for each gamma-class. Gamma-classes assigned a permanent ε-value are excluded from subsequent iterations of FIG. 25, Table 1 (and therefore do not participate in the assignments of the other gamma-classes). The use of the provisional ε-values is discussed at length in 7.\textcircled{1}\textcircled{6}.

7.\textcircled{8}-\textcircled{9}: AASK assigns the an ε-value of 0 (zero) to the permanent ε-value of γ₀, thereby excluding this class and its generative elements from evaluation in subsequent iterations of the Hierarchy Matrix Table 1 in FIG. 25.

AASK then surveys the remaining unassigned delta-classes to determine if any of these entities similarly include the vast majority of the generative elements of the other delta-classes. If any such entities are found to exist (there may be several such delta-classes), they are assigned a permanent ε-value of * (asterisk), and are likewise excluded from further participation in AASK's tabulation of gamma and delta-classes.

7.\textcircled{1}\textcircled{0}: After reducing congruent delta-classes and assigning ε-values to γ₀ and any * classes, AASK is ready to begin the iterative process of assigning ε-values to any remaining gamma-classes.

To facilitate this process AASK maintains several housekeeping fields:
The count of unassigned delta-classes. (δ-classes without a permanent ε-value).
The count of included gamma-classes for each unassigned delta-class.
The number of included gamma-classes in the largest unassigned delta-class
The δₙ designation of the largest unassigned delta-class
The number of gamma-classes included in the largest complement to the largest unassigned delta-class.
The δₙ designation of this largest unassigned complement.
The number of similarly-sized (to within 90%, but this can be parameterized) complements to the largest unassigned delta-class.

Foremost among these values is the count of unassigned delta-classes—as when all classes have been assigned, AASK moves on to formatting its reporting templates. These fields are populated by evaluating the maximum and minimum values in tables 5 and 6 of AASK's Hierarchy Matrix worksheet, as shown in FIG. 25.

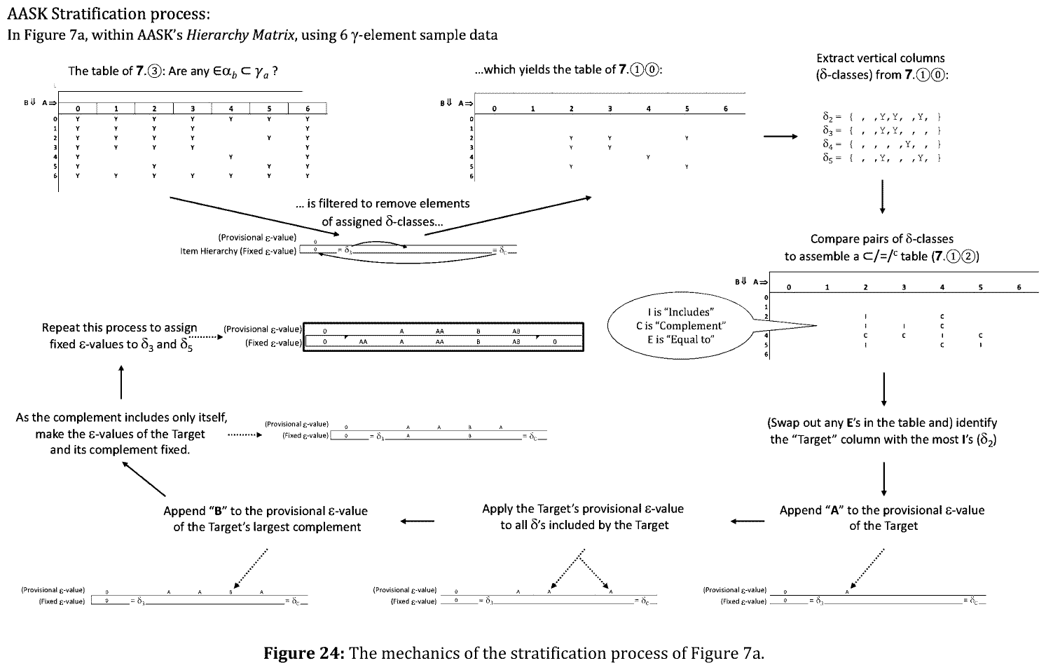

FIG. 24 explores the mechanics of the iterative process of FIG. 7a—employed by AASK to assign ε-values to each gamma-class—using the 6-element sample gamma-classes derived from the 9-element dataset of FIG. 5. When the process of FIG. 24 says to “identify the Target column with the most I's” AASK references Table 5 of FIG. 25, and determines that δ₂ includes three delta-classes, including itself.

Similarly, when the AASK process appends a suffix to “to the temp ε-value of the Target's largest complement” AASK scans the values of column 2 (representing δ₂) of FIG. 25, Table 6 and locates the largest negative value (−1, situated in row 4) to determine that δ₄ is the largest complement of δ₂.

The leftmost column of FIG. 25 illustrates the three operational tables of the Hierarchy Matrix. AASK populates its operational tables by means of iterative/looped routines which (in the case of Table 1) evaluate all paired combinations of (up to) 255 alpha and gamma-class collections. Table 1 is then pruned using the assigned/unassigned permanent 6-values of Table 3 (to exclude rows and columns of data which no longer participate in the stratification process) to create Table 4.

The vertical columns of FIG. 25, Table 4 comprise AASK's revised delta-classes, which are in turn evaluated in pairs as to whether a given delta-class includes, is equal to, or is a complement (⊂/= [symbol] ) of the other delta-classes, forming FIG. 25, Table 2.

Table 5 of FIG. 25 tallies the elements within each delta-class/column of Table 4 (|δₙ|) and the values of this table are employed in creating Table 6, which records the cross-product (vector product) of Tables 2 and 5, whereby the values of Table 5 are indexed by the rows of Table 2 and multiplied by +/−1, depending on whether the base value in Table 2 represents inclusion or complementation.

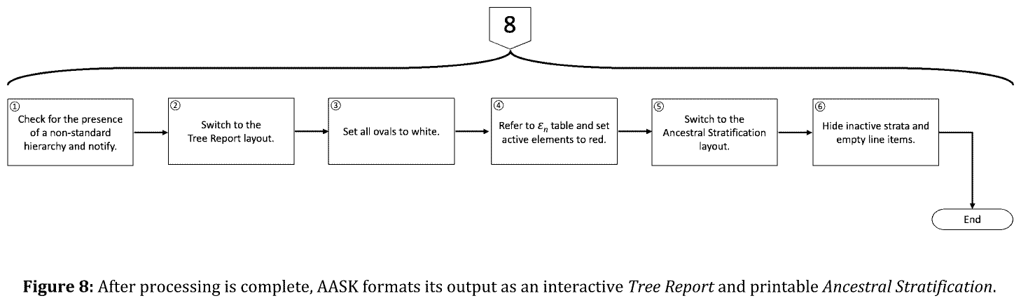

The process of FIG. 7a is repeated until all on have received a permanent ε-value—at which point AASK proceeds onwards to FIG. 8, whereby AASK formats and links these ε-values to its reporting templates.

8.\textcircled{1}: AASK surveys its fully-populated table of positioning vectors to determine if any aspects of the table exceed the parameters of its reporting templates. Such conditions might include: more than 6 generations of the Target Ancestor, or the absence of intermediary ancestral strata, which would cause four complementary delta-classes to be assigned ε-values of A, B, C, and D rather than the hierarchical AA, AB, BA, and BB. AASK notifies the end-user of this status, but inasmuch as such conditions are not fatal, the user is simply advised to consult the Hierarchy Matrix in addition to AASK's reporting templates. Regardless of the limitations of its reporting templates, AASK will present its findings as best it can.

8.\textcircled{2}-\textcircled{4}: AASK's Tree Report displays its hierarchical ε-values in an interactive graphical format. The report template has clickable fields labelled for 6 stratified generations of ε-values, plus fields for AASK's source genetic complex [symbol] ₀ and the supplementary *-class.

FIG. 9 illustrates the functionality of the Tree Report, where White circles indicate unpopulated, inactive ε-values, while Red circles represent populated ε-values. When a populated ε-value is selected, the red circle turns Green, and specific information concerning that class is displayed in two rectangles to the right of the tree layout. The uppermost rectangle displays a boilerplate description of the relationship of the selected class to the Target Ancestor, as a helpful way of maintaining the user's frame of reference—especially where more distantly connected ε-values are concerned. The lower rectangle displays the User Names (blurred in FIG. 9 for privacy reasons) and Test Kit IDs of the individuals (generative elements) assigned to the selected class.

In the case of FIG. 9, the selected ε-class is that of [symbol] ₀, and so the generative elements of [symbol] ₀ displayed in the lower rectangle are the direct descendants of our Target Ancestor which populate α₀.

8.\textcircled{5}-\textcircled{6}: AASK's printable Ancestral Stratification report template is pre-populated with the same ε-classes and descriptive boilerplate text as the Tree Report. AASK's automated routines hide the space allocated to inactive, un-populated ε-classes on the template, in addition to hiding the unused rows within each populated class, as the template supports up to 100 αₙ assignments within each ε-class.

With its reports formatted, AASK returns to the Tree Report layout and terminates execution.

### III. AASK at the Enterprise Level

AASK may be performed at the Enterprise level by deploying relational data structures in a manner consistent with the tables employed by the AASK Engine on the desktop platform. The specific methodologies and techniques required to add AASK functionality to an existing genealogical database will necessarily depend on the DBMS (database management system) used, but the general framework outlined in this section should provide adequate guidance to the experienced programmer.

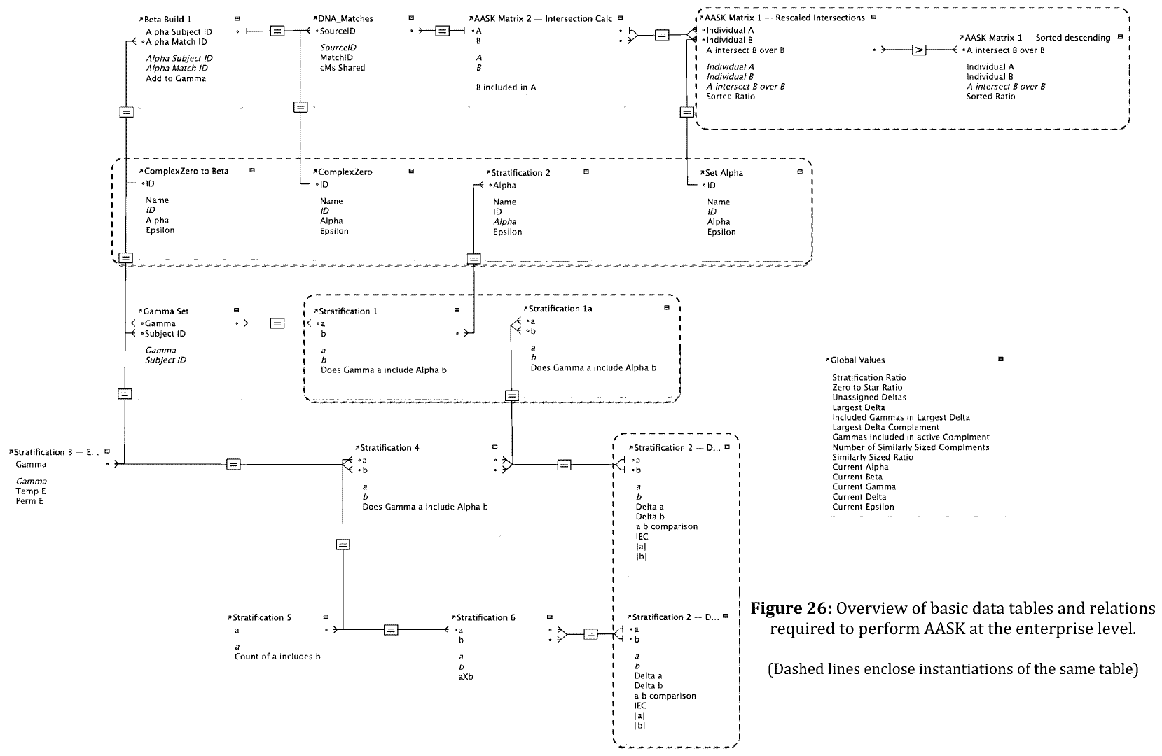

FIG. 26 provides a basic overview of the data tables required to perform AASK at the Enterprise level. Instantiations of the same table are enclosed together by a dashed line. Data structures are indicated in courier type. Unless prefixed with a new [Table: Field] format, :Fields listed in the same paragraph with an empty or absent table prefix may be assumed to be from the table referenced at the start of the paragraph. As with Section II, the numbering of processes in the previously presented flowcharts are maintained in the following description of the structure and operation of AASK at the Enterprise level.

While the AASK process remains unchanged, the logistics of operating within a database management system (DBMS) necessitate a number of changes as to how AASK's input data is formatted. Rather than pasting the elements of [symbol] ₀ in the first position of the Beta-build worksheet and storing the generative elements of this complex elsewhere, on the Gamma-model, the Subject Name and Test Kit ID for each element of [symbol] ₀ are stored in the Complex Zero table, along with the Subject Name and Test Kit ID of each of the generative elements of the complex.

From the outset of the process within a DBMS, the generative elements themselves are differentiated from the elements of [symbol] ₀ proper by assigning zero (0) to the Complex Zero:Alpha (α) and :Epsilon (ε) fields of the generative elements only. Because the beta, gamma, and delta meta-classes all correspond 1-to-1 with their assigned alpha-class, there is no need for separate fields to record these categories of data. However, since the epsilon meta-class uses an A/B ordered binary to specify the hierarchical position of each alpha-class, these values are recorded in a separate field in the Complex Zero table.

AASK uses the set of DNA matches for each individual element of [symbol] ₀, and these values are all stored in a single table, (DNA Matches) which includes a courtesy field for :cMs Shared, although these values are neither required nor utilized by AASK itself.

AASK's “housekeeping” fields, and several parameterized values used to fine-tune the process, and variables otherwise required for the successful implementation of AASK, are relegated to the Global Values table, so as to be accessible to all other tables in the DBMS.

All relations shown within FIG. 26 are based on equivalence with the exception of the relation that facilitates the ratio of sorted elements from process 5.\textcircled{4}, which returns the next smallest element, as required by process 5.\textcircled{4}. Apart from these considerations, the AASK process itself remains largely invariant whether implemented on a desktop platform via the AASK Engine or within a DBMS.

1. A process for performing Axiomatic Ancestral Stratification by Kinship (AASK) of autosomal DNA (atDNA) matches, independent of any specific testing provider or tabulating mechanism.

2. The process of claim 1, where a genetic complex, [symbol] ₀, obtained via the CMA process (U.S. application Ser. No. 17/470,321) and the generative elements of said complex, are logically compounded with the autosomal DNA (atDNA) matches of each element of this genetic complex.

3. The process of claim 1, where the totality of a nexus individual's autosomal DNA (atDNA) matches are logically compounded with the atDNA matches of each individual who matches the nexus, without any CMA preprocessing.

4. The process of claim 1, whereby the test subject elements of [symbol] ₀ are grouped into meta-classes—collections of elements of [symbol] ₀ and elements taken from the atDNA matches of the elements of [symbol] ₀—such that there exists: The alpha-class (αₙ), where selected elements of [symbol] ₀ share a common line of descent relative to the generative elements of [symbol] ₀; The beta-class (βₙ), consisting of the In Common With (ICW) matches of the elements of a given alpha-class; The gamma-class (γₙ), consisting of elements common to [symbol] ₀ and a given beta-class; The delta-class (δₙ), an ordered set derived from a survey of whether a given γₙ includes any elements of other alpha-classes yet to receive their εₙ designation; The epsilon-class (εₙ), a positioning vector that locates the elements of a given αₙ collection within the hierarchy of AASK's reporting structure.

5. The process of claim 1, whereby the creation of the above meta-classes has been automated through the application of set-theoretic axioms and procedures.

6. The process of claim 1, wherein elements of a genetic complex are grouped by common lines of descent using their degree of mutual set-theoretic inclusion.

7. The process of claim 1, wherein delta-classes are iteratively re-evaluated in light of each generation's assigned positioning vectors.

8. The process of claim 1, wherein pairs of delta-classes are iteratively re-evaluated as to whether they include or complement each other.

9. The process of claim 1, wherein the cross product (vector product) of the cardinality of each delta-class (|δₙ|) and the inclusion/complementation of other delta-classes is used to identify: the delta-class with the greatest degree of mutual inclusion, and the largest complements of that delta-class.

10. The process of claim 1, wherein a unique provisional positioning vector is assigned to a “target” delta-class with greatest mutual inclusion and to the delta-classes included therein.

11. The process of claim 1, wherein hierarchical positioning vectors are expressed as an ordered (A/B) binary, supplemented by the 0 and * classes.

12. The process of claim 1, wherein a unique provisional positioning vector is assigned to each instance of the largest complements of the “target” delta-class and to the delta-classes included therein.

13. The process of claim 1, whereby the (A/B) system of hierarchical vectors may be supplemented with additional letters in order to accommodate imperfect or incomplete generational hierarchies.

14. The process of claim 1, wherein hierarchically organized alpha-classes are interactively presented in a report alongside actionable intelligence pertaining to the alpha-classes' genealogical relationship to the Target Ancestor of their genetic complex.

15. The process of claim 1, wherein the hierarchy of alpha-classes is also presented in a print-friendly report containing the same actionable intelligence.

16. Scripted spreadsheet implementations of the process of claim 1.

17. A DBMS (Database Management System) implementation of the process of claim 1.

18. The DBMS implementation of claim 17, wherein AASK-specific data tables and methods are appended to an existing genealogical DBMS.
REFERENCED CITED
U.S. Patent Documents

| | Priority | Publication | | |
|---|---|---|---|---|
| Publication # | Date | Date | Assignee | Title |
| 20230077642A1 | 2021 Sep. 9 | 2023 Mar. 17 | Arun Konanur | Systems And Methods for |
| | | | | Performing Correlated |
| | | | | Multiphasic Analysis |
| 20170213127A1 | 2016 Jan. 24 | 2017 Jul. 27 | Matthew Charles | Method and System for |
| | | | Duncan | Discovering Ancestors using |
| | | | | Genomic and Genealogic Data |
| 20180189379A1 | 2016 Dec. 29 | 2018 Jul. 05 | Ancestry.Com | Dynamically-qualified aggregate |
| | | | Operations Inc. | relationship system in |
| | | | | genealogical databases |
| 10720229B2 | 2014 Oct. 14 | 2020 Jul. 21 | Ancestry.Com | Reducing error in predicted |
| | | | DNA, LLC | genetic relationships |
| 8738297B2 | 2001 Mar. 30 | 2014 May 27 | Ancestry.Com | Method for molecular |
| | | | DNA, LLC | genealogical research |
| 20060025929A1 | 2004 Jul. 30 | 2006 Feb. 2 | Chris Eglington | Method of determining a genetic |
| | | | | relationship to at least one |
| | | | | individual in a group of famous |
| | | | | individuals using a combination |
| | | | | of genetic markers |
| 20090118131A1 | 2008 Oct. 15 | 2009 May 7 | 23andme Inc. | Genetic comparisons between |
| | | | | grandparents and |
| | | | | grandchildren |
| 20140006433A1 | 2013 Apr. 26 | 2014 Jan. 2 | 23andme Inc. | Finding relatives in a database |
| 20140067355A1 | 2013 Sep. 6 | 2014 Mar. 6 | Ancestry.Com | Using Haplotypes to Infer |
| | | | DNA, LLC | Ancestral Origins for Recently |
| | | | | Admixed Individuals |
| 20140108527A1 | 2012 Oct. 17 | 2014 Apr. 17 | Fabric Media Inc | Social genetics network for |
| | | | | providing personal and business |
| | | | | services |
| 20140278138A1 | 2013 Mar. 15 | 2014 Sep. 18 | Ancestry.Com | Family Networks |
| | | | DNA, LLC | |
| 8855935B2 | 2006 Oct. 2 | 2014 Oct. 7 | Ancestry.Com | Method and system for |
| | | | DNA, LLC | displaying genetic and |
| | | | | genealogical data |
| 20140067280A1 | 2012 Aug. 28 | 2014 Mar. 6 | Inova Health | Ancestral-Specific Reference |
| | | | System | Genomes And Uses Thereof |

Foreign Patent Documents

| Publication # | Priority Date | Publication Date | Asignee | Title |
|---|---|---|---|---|
| WO2019217574A1 | 2018 May 8 | 2019 Nov. 14 | Ancestry.Com | Genealogy item ranking and |
| | | | Operations Inc. | recommendation |
| WO2020018991A1 | 2018 Jul. 20 | 2020 Jan . 23 | Ancestry.Com | System and method for |
| | | | Operations Inc. | genealogical entity resolution |
| WO2020257166A1 | 2019 Jun. 17 | 2020 Dec. 24 | Ancestry. Com | Genealogical tree tracing and |
| | | | Operations Inc. | story generation |
| WO2021051018A1 | 2019 Sep. 13 | 2021 Mar. 18 | 23andme, Inc. | Methods and systems for |
| | | | | determining and displaying |
| | | | | pedigrees |
| WO2000018960A3 | 1998 Sep. 25 | 2000 Sep. 08 | Ancestry.Com | Methods and products related |
| | | | DNA, LLC | to genotyping and DNA analysis |
| WO2009051766A1 | 2007 Oct. 15 | 2009 Apr. 23 | 23andme, Inc. | Family inheritance |
